(19) United States
(12) Patent Application Publication
(10) Pub. No.: US 2022/0144840 A1
(43) Pub. Date: May 12, 2022

(54) HETEROCYCLIC COMPOUNDS AND THEIR USE IN THE TREATMENT OF AMYLOID-RELATED DISEASES

(71) Applicant: D.E. Shaw Research, LLC, New York, NY (US)

(72) Inventors: Fabrizio GIORDANETTO, New York, NY (US); Cecily CAMPBELL-BEZAT, New York, NY (US); Albert PAN, New York, NY (US)

(21) Appl. No.: 17/429,115

(22) PCT Filed: Feb. 6, 2020
(86) PCT No.: PCT/US2020/016934

§ 371 Date: Aug. 6, 2021

Related U.S. Application Data
(60) Provisional application No. 62/803,663, filed on Feb. 11, 2019.

(51) Int. Cl. C07D 487/04; C07D 471/04

(52) U.S. Cl. CPC C07D 487/04; C07D 471/04

(57) ABSTRACT

A compound of Formula (I) or a pharmaceutically acceptable salt thereof is described, wherein the substituents are as defined herein. Pharmaceutical compositions comprising the same and method of using the same are also described.

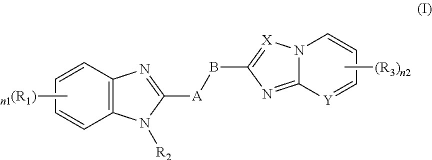

This application claims the benefit of U.S. Provisional Application No. 62/803,663, filed Feb. 11, 2019, the entire content of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and/or compositions useful as pharmaceuticals in the treatment of amyloid-related diseases.

### BACKGROUND

An increasing number of age-related and degenerative diseases are associated with misfolding-induced protein aggregation. Peptides that misfold into β-strands have a propensity to form β-sheets via hydrogen bonding, which, in turn, oligomerize to form soluble β-structured aggregates. Supramolecular association of these aggregates generates insoluble β-cross-sectional amyloid fibrils. Fibrils commonly deposit extracellularly as amyloid plaques or, less commonly, as intracellular inclusions.

It has been reported that the amyloid aggregation process is an equilibrium (Dobson, C. M. et al., 2003, Nature, 426:884-890). Soluble oligomeric species formed en route to fibrils/deposits and those liberated from already formed fibrils/deposits are cytotoxic through a variety of pathologic pathways including inducing membrane damage, oxidative stress, and inflammation. Fibrils and deposits can also trigger oligomer release, impair protein homeostasis, and/or nonspecifically overwhelm their resident tissues leading to organ failure.

Amyloid or amyloid-like oligomers, fibrils, and plaques (hereafter collectively termed “amyloids”) are implicated in the pathogenesis and progression of a growing number of diseases. These diseases, or amyloidoses, can be characterized, in part, by the location of the associated amyloids. In general, amyloids localized in the central nervous system (CNS) are linked to neurodegenerative conditions, such as Alzheimer's disease (AD). Those amyloids localized to specific organs (e.g., the eye) are linked to diseases of these organs (e.g., macular degeneration). The amyloids present in multiple organs are linked to a wide range of systemic amyloidoses.

AD is the prototypical example of a neurodegenerative condition characterized by the formation and growth of amyloid plaques. In most AD cases, these plaques arise from the aggregation of amyloid beta (Aβ). Aβ is cleaved as a 37-49-residue peptide from the constitutively expressed amyloid precursor protein by β- and γ-secretases. Endocytic processing further cleaves Aβ to 40- and 42-amino acid peptides. These peptides, Aβ40 and Aβ42, are the primary monomers that form soluble Aβ oligomers. Oligomers then assemble into insoluble Aβ fibrils, which are ultimately deposited as extracellular Aβ plaques in brain tissue. There are various interconnected mechanisms by which these Aβ species contribute to the initiation and progression of AD. Aβ oligomers can initiate a cascade of toxic insults to neural cells, including reactive oxygen species and inflammation. The Aβ oligomers are also linked to hyperphosphorylation of the microtubule-associated protein tau (τ), leading to cytoskeletal collapse. Once formed, fibrils and plaques are also pathogenic, either directly or indirectly through release of toxic Aβ oligomers. The net result of these pathogenic effects is loss of brain tissue and disruption of neurotransmission.

There are currently no preventative or curative treatments for AD. Palliative treatments, such as the acetylcholinesterase inhibitors, have shown modest efficacy for managing some cognitive and behavioral symptoms, but have no effect on disease progression and outcome. Further, these drugs often have significant adverse effects. Aβ has been considered as a pharmacological target for AD (Cummings, J. et al., 2018, Alzheimers Dement (NY), 4:195-214). Such efforts will be aided by recent advances in structural characterization of Aβ monomers, oligomers, and aggregates (Meier, B. H. et al., 2017, Trends Biochem. Sci., 42:777-787).

Many other neurodegenerative conditions are associated with amyloids and described below. Down syndrome, hereditary cerebral hemorrhage with amyloidosis (HCHWA, Dutch type), cerebral amyloid angiopathy, cerebrovascular type dementia, mild cognitive impairment, multiple sclerosis, and senile dementia are all associated with Aβ aggregation. Aggregation of α-synuclein is associated with Parkinson's disease, dementia with Lewy bodies (insoluble inclusions), and multiple system atrophy. Creutzfeldt-Jakob disease, Gerstmann-Straussler-Scheinker disease, fatal familial insomnia, bovine spongiform encephalopathy (in cows), possibly all forms of transmissible encephalopathy, scrapie (in sheep), and kuru have been shown to be associated with aggregation of prion proteins. This aggregation of prion proteins, when compounded with aggregation of Huntington exon I, may also result in Huntington's disease. The aggregation of proteins and peptides with extended glutamine repeats is associated with dentatorubral pallidoluysian atrophy, spinal and bulbar muscular atrophy, spinocerebellar ataxia (types 1, 2, 3, 6, and 7), and other inheritable neurodegenerative diseases. Guam Parkinson dementia complex, Pick's disease, progressive supranuclear palsy, and frontotemporal dementia have been shown to be associated with τ aggregation. Familial British dementia and familial Danish dementia are associated with aggregation of the peptides ABri and ADan, respectively. Aggregation of Cu—Zn superoxide dismutase is associated with amyotrophic lateral sclerosis and, possibly, other motor neuron diseases. Hereditary cerebral hemorrhage with amyloidosis (HCHWA, Icelandic type) is associated with aggregation of N-terminal truncated cystatin C. Aggregation of certain viral proteins is associated with HIV-related dementia.

Protein aggregation in or across various major organs (e.g., kidneys, heart, liver, spleen, and gastrointestinal (GI) tract) is also implicated in the causation or progression of several non-CNS systemic amyloidoses, examples of which are described below. Inclusion body myositis and certain endocrine tumors are associated with Aβ aggregation. Dialysis-related amyloidosis is associated with aggregation of β₂-microglobulin in bones, joints, and tendons. Prostatic amyloid has been shown to be associated with aggregation of β₂-microglobulin in the prostate. Aggregation of immunoglobulin light or heavy chains in the kidneys, heart, liver, and GI tract may result in myeloma-associated amyloidosis, primary systemic amyloidosis, and systemic and nodular AL amyloidoses. Aggregation of serum amyloid A protein and deposition in the liver, kidneys, and spleen are associated with secondary systemic amyloidosis, familial Mediterranean fever, reactive systemic AA amyloidosis, and chronic inflammatory disease. Hereditary non-neuropathic systemic amyloidosis and familial visceral amyloidosis are associated with insoluble deposits/aggregates of mutant lysozyme in the liver, kidneys, and spleen. Fibrinogen α-chain amyloidosis is associated with aggregation of the fibrinogen A α-chain and subsequent deposition in the liver and kidneys. Aggregation of gelsolin in the cornea is associated with Finnish hereditary systemic amyloidosis. Senile systemic amyloidosis and familial amyloid cardiomyopathy (FAC) are primarily linked to transthyretin aggregates in the heart. Familial amyloid polyneuropathy (FAP) is also linked to such aggregates, but primarily in the peripheral nerves. Transthyretin aggregates found in other organs (e.g., GI tract) also contribute to senile systemic amyloidosis, familial amyloid polyneuropathy, and familial amyloid cardiomyopathy. FAP type II is linked to aggregation of apolipoprotein AI in peripheral nerves. Medullary carcinoma of the thyroid has been shown to be associated with calcitonin aggregation. The aggregation of atrial natriuretic peptide in the heart has been implicated in isolated atrial amyloidosis. Aggregation of the 37-residue islet amyloid polypeptide (also called amylin) is implicated in type II diabetes via progressive destruction of insulin-producing β cells in the islets of Langerhans cells in the pancreas. Injection-site aggregation of insulin in diabetic patients may cause insulin-related amyloidosis.

Amyloids are also implicated in the causation and/or progression of ocular diseases. Macular degeneration (MD), particularly age-related MD (AMD), is associated with deposits of extracellular drusen in the macula of the retina. The accumulation of drusen, a yellow deposit of proteins and lipids that contains Aβ, is believed to be implicated in the progressive breakdown of macular cell layers that ultimately damage the retina. This retinal degradation is termed dry AMD, the stage (early, intermediate, and late) of which bears positive correlation to drusen size and quantity. Dry AMD can evolve into advanced wet AMD at any stage, which results in rapid damage to the macula due to leaky vasculature behind the macula. Wet AMD can be managed using antibodies targeting vascular growth factors, laser photocoagulation, and photodynamic therapy, but the results are mixed and lost vision is not restored. Other ocular diseases are associated with an Aβ-related pathological abnormality or change in the tissue of the visual system. These include, for example, cortical visual deficits, glaucoma, ocular amyloidosis, primary retinal degeneration, optic neuropathy, optic neuritis, lattice dystrophy, and cataracts.

Thus, there remains a need for novel pharmaceutical agents capable of inhibiting, preventing, and/or reversing amyloids and the related aggregation process.

### SUMMARY OF THE INVENTION

In one aspect, compounds useful as amyloid inhibitors having a structure of Formula

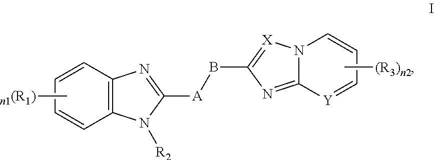

are described, where the various substituents are defined herein. The compounds of Formula I described herein inhibit the formation of amyloid and can be used in the treatment of a variety of amyloid-related conditions. Also described herein are pharmaceutical compositions including the compounds of Formula I and methods of using these compositions or compounds described herein for treating amyloid-related conditions and/or preventing amyloid formation in vitro or in vivo. Methods for synthesizing these compounds are also described herein.

The compounds, pharmaceutical compositions, and methods of treatment described herein have a number of clinical applications, including as pharmaceutically active agents and methods for treating Alzheimer's disease, mild cognitive impairment, senile dementia, Down syndrome, cerebral amyloid angiopathy, inclusion body myositis, hereditary cerebral hemorrhage with amyloidosis (Dutch type), the Guam Parkinson-Dementia complex, macular degeneration, fronto-temporal dementia, Parkinson's disease, dementia with Lewy bodies, cerebrovascular type dementia, Pick's disease, Huntington's disease, dentatorubral pallidoluysian atrophy, spinocerebellar ataxia (SCA, types 1, 2, 3, 6, and 7), spinal and bulbar muscular atrophy, Creutzfeldt-Jakob disease, bovine spongiform encephalopathy in cows, scrapie in sheep, kuru, Gerstmann-Straussler-Scheinker disease, fatal familial insomnia, amyotrophic lateral sclerosis, familial British dementia, familial Danish dementia, hereditary cerebral hemorrhage with amyloidosis (HCHW A, Icelandic type), type II diabetes, dialysis-related amyloidosis, prostatic amyloid, primary systemic amyloidosis, systemic AL amyloidosis, nodular AL amyloidosis, myeloma associated amyloidosis, systemic (reactive) AA amyloidosis, secondary systemic amyloidosis, chronic inflammatory disease, familial Mediterranean fever, senile systemic amyloidosis, familial amyloid polyneuropathy, familial cardiac amyloid, familial visceral amyloidosis, hereditary non-neuropathic systemic amyloidosis, Finnish hereditary systemic amyloidosis, fibrinogen α-chain amyloidosis, insulin-related amyloidosis, medullary carcinoma of the thyroid, isolated atrial amyloidosis, cataract, progressive supranuclear palsy, multiple sclerosis, HIV-related dementia, senile cardiac amyloidosis, endocrine tumors, neuronal degradation, cortical visual deficits, glaucoma, ocular amyloidosis, primary retinal degeneration, optic nerve drusen, optic neuropathy, optic neuritis, lattice dystrophy, and a combination thereof.

In one aspect, a compound of Formula I or a pharmaceutically acceptable salt thereof is described,

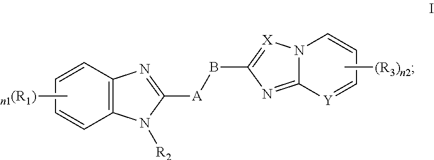

wherein

each occurrence of R₁ is independently H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}; or alternatively two R₁ groups and the carbon atoms they are connected to taken together form a 4-7 membered carbocycle or heterocycle optionally substituted by one or more alkyl, halogen, ORₐ, or oxo;

R₂ is H, alkyl, heteroalkyl, cycloalkyl, or cycloheteroalkyl;

-A-B- is —S—CR₄R₅— or —CR₄R₅—S—;

R₄ and R₅ are each independently H, alkyl, or cycloalkyl; or alternatively R₄, R₅ and the carbon atom they are connected to taken together form a 3-7 membered carbocycle or heterocycle optionally substituted by one or more alkyl, halogen, ORₐ, or oxo;

X is N or CR₃;

Y is N or CR₃;

each occurrence of R₃ is independently H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}; or alternatively two R₃ groups and the carbon atoms they are connected to taken together form a 4-7 membered carbocycle or heterocycle optionally substituted by one or more alkyl, halogen, ORₐ, or oxo;

each occurrence of Rₐ and R_{b} are independently H, alkyl, cycloalkyl, optionally substituted saturated heterocycle, optionally substituted aryl, or optionally substituted heteroaryl; or alternatively Rₐ and R_{b} together with the nitrogen atom that they are connected to form a heterocycle comprising the nitrogen atom and 0-3 additional heteroatoms each selected from the group consisting of N, O, and S and optionally substituted by one or more alkyl, halogen, ORₐ, or oxo;

n₁ is an integer from 0-4; and

n₂ is an integer from 0-3;

with the proviso that the compound of Formula I is not H or

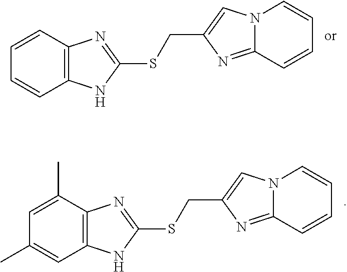 [SMILES: c1ccc2[nH]c(SCc3cn4ccccc4n3)nc2c1 ; Cc1cc(C)c2nc(SCc3cn4ccccc4n3)[nH]c2c1]

In any one of the embodiments described herein, n₁ is 0, 1, or 2.

In any one of the embodiments described herein, at least one occurrence of R₁ is halogen or NO₂.

In any one of the embodiments described herein, at least one occurrence of R₁ is F, Cl, or NO₂.

In any one of the embodiments described herein, at least one occurrence of R₁ is F or Cl.

In any one of the embodiments described herein, at least one occurrence of R₁ is H, alkyl, halogenated alkyl, cycloalkyl, ORₐ, CN, or (C═O)OR_{b}.

In any one of the embodiments described herein, at least one occurrence of R₁ is NRₐR_{b}, NRₐ(C═O)R_{b}, or CONRₐR_{b}.

In any one of the embodiments described herein, R₂ is H, alkyl, or cycloalkyl.

In any one of the embodiments described herein, R₂ is H, CH₃, or CH₂CH₃.

In any one of the embodiments described herein, R₂ is heteroalkyl or cycloheteroalkyl.

In any one of the embodiments described herein, -A-B- is —S—CR₄R₅—.

In any one of the embodiments described herein, -A-B- is —CR₄R₅—S—.

In any one of the embodiments described herein, at least one of R₄ and R₅ is H or alkyl.

In any one of the embodiments described herein, CR₄R₅ is CH₂, CHCH₃, or C(CH₃)₂.

In any one of the embodiments described herein, at least one of R₄ and R₅ is cycloalkyl.

In any one of the embodiments described herein, X may be N.

In any one of the embodiments described herein, X may be CR₃.

In any one of the embodiments described herein, Y may be N.

In any one of the embodiments described herein, Y may be CR₃.

In any one of the embodiments described herein, X and Y may both be N.

In any one of the embodiments described herein, X and Y may both be CR₃.

In any one of the embodiments described herein, at least one occurrence of R₃ is H, alkyl, halogenated alkyl, or halogen.

In any one of the embodiments described herein, at least one occurrence of R₃ is H, CH₃, CH₂CH₃, F, Cl, or Br.

In any one of the embodiments described herein, at least one occurrence of R₃ is cycloalkyl, ORₐ, CN, (C═O)OR_{b}, or NO₂.

In any one of the embodiments described herein, at least one occurrence of R₃ is NRₐR_{b}, NRₐ(C═O)R_{b}, or CONRₐR_{b}.

In any one of the embodiments described herein, n₂ is 0, 1, or 2.

In any one of the embodiments described herein, at least one of Rₐ and R_{b} is H, alkyl, or cycloalkyl.

In any one of the embodiments described herein, at least one of Rₐ and R_{b} is H, CH₃, CH₂CH₃, propyl, isopropyl, cyclopropyl, or cyclobutyl.

In any one of the embodiments described herein, at least one of Rₐ and R_{b} is optionally substituted saturated heterocycle, optionally substituted aryl, or optionally substituted heteroaryl.

In any one of the embodiments described herein, Rₐ and R_{b} together with the nitrogen atom that they are connected to form an optionally substituted heterocycle comprising the nitrogen atom and 0-3 additional heteroatoms each selected from the group consisting of N, O, and S.

In any one of the embodiments described herein, the structural moiety

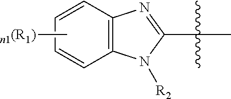

has the structure of

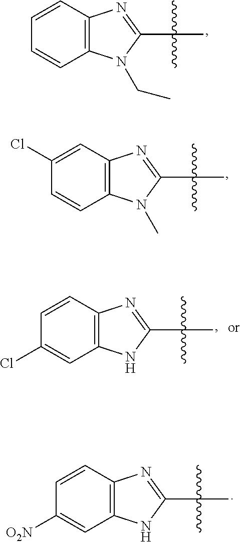

In any one of the embodiments described herein, the structural moiety

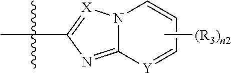

has the structure of

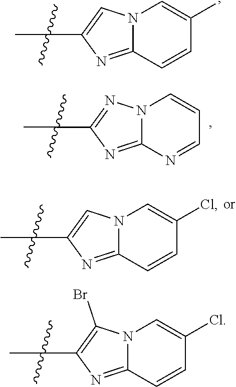

In any one of the embodiments described herein, the compound is selected from the group consisting of

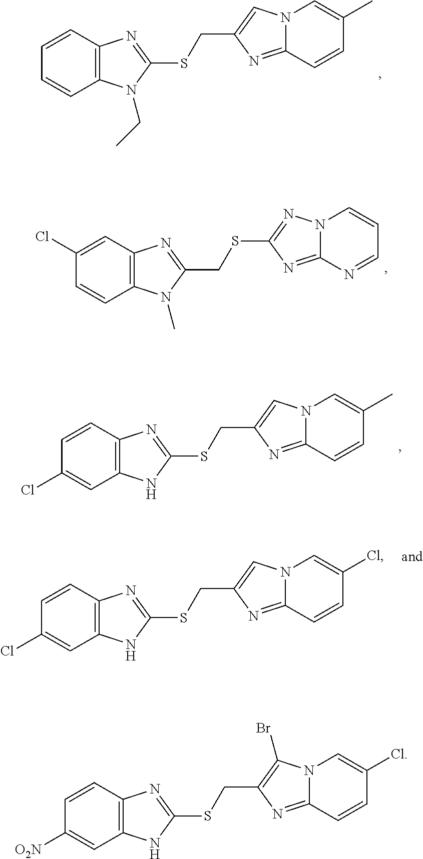 [SMILES: CCn1c(SCc2cn3cc(C)ccc3n2)nc2ccccc21 ; Cn1c(CSc2nc3ncccn3n2)nc2cc(Cl)ccc21 ; Cc1ccc2nc(CSc3nc4ccc(Cl)cc4[nH]3)cn2c1 ; Clc1ccc2nc(SCc3cn4cc(Cl)ccc4n3)[nH]c2c1 ; O=[N+]([O-])c1ccc2nc(SCc3nc4ccc(Cl)cn4c3Br)[nH]c2c1]

In another aspect, a pharmaceutical composition is disclosed, including at least one compound according to any one of the embodiments disclosed herein or a pharmaceutically acceptable salt thereof and a pharmaceutically acceptable carrier or diluent.

In yet another aspect, a method of treating an amyloid-related disease in a mammalian species in need thereof is disclosed, including administering to the mammalian species a therapeutically effective amount of at least one compound according to any one of the embodiments disclosed herein or a pharmaceutically acceptable salt thereof.

In any one of the embodiments described herein, the amyloid-related disease is selected from the group consisting of Alzheimer's disease, mild cognitive impairment, senile dementia, Down syndrome, cerebral amyloid angiopathy, inclusion body myositis, hereditary cerebral hemorrhage with amyloidosis (Dutch type), the Guam Parkinson-Dementia complex, macular degeneration, fronto-temporal dementia, Parkinson's disease, dementia with Lewy bodies, cerebrovascular type dementia, Pick's disease, Huntington's disease, dentatorubral pallidoluysian atrophy, spinocerebellar ataxia (SCA, types 1, 2, 3, 6, and 7), spinal and bulbar muscular atrophy, Creutzfeldt-Jakob disease, bovine spongiform encephalopathy in cows, scrapie in sheep, kuru, Gerstmann-Straussler-Scheinker disease, fatal familial insomnia, amyotrophic lateral sclerosis, familial British dementia, familial Danish dementia, hereditary cerebral hemorrhage with amyloidosis (HCHW A, Icelandic type), type II diabetes, dialysis-related amyloidosis, prostatic amyloid, primary systemic amyloidosis, systemic AL amyloidosis, nodular AL amyloidosis, myeloma associated amyloidosis, systemic (reactive) AA amyloidosis, secondary systemic amyloidosis, chronic inflammatory disease, familial Mediterranean fever, senile systemic amyloidosis, familial amyloid polyneuropathy, familial cardiac amyloid, familial visceral amyloidosis, hereditary non-neuropathic systemic amyloidosis, Finnish hereditary systemic amyloidosis, fibrinogen α-chain amyloidosis, insulin-related amyloidosis, medullary carcinoma of the thyroid, isolated atrial amyloidosis, cataract, progressive supranuclear palsy, multiple sclerosis, HIV-related dementia, senile cardiac amyloidosis, endocrine tumors, neuronal degradation, cortical visual deficits, glaucoma, ocular amyloidosis, primary retinal degeneration, optic nerve drusen, optic neuropathy, optic neuritis, lattice dystrophy, and a combination thereof.

In any one of the embodiments described herein, the macular degeneration is age-related macular degeneration.

In any one of the embodiments described herein, the amyloid-related disease is a neurodegenerative disorder.

In any one of the embodiments described herein, the neurodegenerative disorder is selected from the group consisting of Alzheimer's disease, Parkinson's disease, dementia with Lewy bodies, Huntington's disease, cerebrovascular type dementia, Down syndrome, hereditary cerebral hemorrhage with amyloidosis (Dutch type), the Guam Parkinson-Dementia complex, mild cognitive impairment, Pick's disease, Creutzfeldt-Jakob disease, amyotrophic lateral sclerosis, and a combination thereof.

In any one of the embodiments described herein, the amyloid-related disease is an ocular disease associated with a β-amyloid-related pathological abnormality or change in the tissue of the visual system.

In any one of the embodiments described herein, the ocular disease is selected from the group consisting of cortical visual deficits, glaucoma, cataract due to β-amyloid deposition, ocular amyloidosis, primary retinal degeneration, macular degeneration, optic nerve drusen, optic neuropathy, optic neuritis, and lattice dystrophy.

In any one of the embodiments described herein, the mammalian species is human.

In yet another aspect, a method of retaining or increasing cognitive memory capacity in a mammalian species suffering from memory impairment is disclosed, including administering to the mammalian species a therapeutically effective amount of at least one compound according to any one of the embodiments disclosed herein or a pharmaceutically acceptable salt thereof.

In any one of the embodiments described herein, the mammalian species is human.

In any one of the embodiments described herein, a method of reducing the β-amyloid plaque load, inhibiting the formation of β-amyloid plaques, and/or retarding the increase of amyloid load in the brain in a mammalian species in need thereof is disclosed, including administering to the mammalian species a therapeutically effective amount of at least one compound according to any one of the embodiments disclosed herein or a pharmaceutically acceptable salt thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited, to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle (including heteroaryl), and aryl can themselves be optionally substituted.

The term “heteroalkyl” refers to a straight- or branched-chain alkyl group preferably having from 2 to 12 carbons, more preferably 2 to 10 carbons in the chain, one or more of which has been replaced by a heteroatom selected from the group consisting of S, O, P, and N. Exemplary heteroalkyls include, but are not limited to, alkyl ethers, secondary and tertiary alkyl amines, alkyl sulfides, and the like. The group may be a terminal group or a bridging group.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited, to one or more of the following groups: hydrogen, halogen, alkyl, halogenated alkyl, (i.e., an alkyl group bearing single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, and hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “heterocycloalkyl” or “cycloheteroalkyl” refers to a saturated or partially saturated monocyclic, bicyclic, or polycyclic ring containing at least one heteroatom selected from the group consisting of nitrogen, sulfur, and oxygen, preferably from 1 to 3 heteroatoms in at least one ring. Each ring is preferably from 3 to 10 membered, more preferably 4 to 7 membered. Examples of suitable heterocycloalkyl substituents include, but are not limited to, pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane. The group may be a terminal group or a bridging group.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}Rₑ, OC(═O)Rₐ, OC(═O)NR_{b}Rₑ, NR_{b}C(═O)ORₑ, NRₐC(═O)NR_{b}Rₑ, NR_{d}S(═O)₂NR_{b}Rₑ, NRₐP(═O)₂NR_{b}Rₑ, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl or naphthyl. The term “carbocycle” encompasses cycloalkyl, cycloalkenyl, cycloalkynyl and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. The term “heteroarylium” refers to a heteroaryl group bearing a quaternary nitrogen atom and thus a positive charge. The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

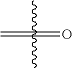 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be re-arranged to satisfy the valence requirement. For instance, a pyridine with a 2-oxo substituent group may have the structure of

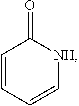

which also includes its tautomeric form of

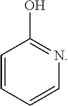

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cycloalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,3,4-triazinolyl and tetrazolyl.

The terms “halogen” or “halo” refer to chlorine, bromine, fluorine or iodine.

The term “substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl propyl and butyl chlorides bromides, and iodides), dialkyl sulfates (e.g., dimethyl, diethyl, dibutyl and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios or isomer ratios in a range bounded by any two isomer ratios described herein are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements and, hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. It will be further appreciated that the substituents (e.g., alkyl, cycloalkyl, aryl, heteroaryl, heterocycle), as described herein, may themselves be substituted with any number of substituents or functional moieties whether or not the term “optionally substituted” is used to describe the substituents. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “amyloid” refers to the misfolding and aggregation of proteins or peptides into insoluble fibrils. In humans, amyloid formation has been linked to a plurality of diseases. As used herein, the term “amyloid-related” disease or condition refers to a disease or condition linked to or caused by the formation of amyloid.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as amyloid inhibitors are described. Applicants have surprisingly found that the compounds disclosed herein exhibit potent activities inhibiting the formation of amyloid and therefore can be used in the treatment of various amyloid-related conditions.

In one aspect, a compound of Formula I or a pharmaceutically acceptable salt thereof is described,

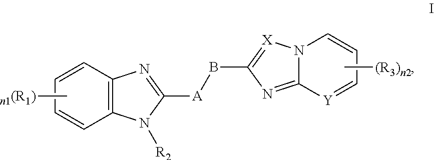

wherein

each occurrence of R₁ is independently H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}; or alternatively two R₁ groups and the carbon atoms they are connected to taken together form a 4-7 membered carbocycle or heterocycle optionally substituted by one or more alkyl, halogen, ORₐ, or oxo;

R₂ is H, alkyl, heteroalkyl, cycloalkyl, or cycloheteroalkyl;

-A-B- is —S—CR₄R₅— or —CR₄R₅—S—;

R₄ and R₅ are each independently H, alkyl, or cycloalkyl; or alternatively R₄, R₅ and the carbon atom they are connected to taken together form a 3-7 membered carbocycle or heterocycle optionally substituted by one or more alkyl, halogen, ORₐ, or oxo;

X is N or CR₃;

Y is N or CR₃;

each occurrence of R₃ is independently H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}; or alternatively two R₃ groups and the carbon atoms they are connected to taken together form a 4-7 membered carbocycle or heterocycle optionally substituted by one or more alkyl, halogen, ORₐ, or oxo;

each occurrence of Rₐ and R_{b} are independently H, alkyl, cycloalkyl, optionally substituted saturated heterocycle, optionally substituted aryl, or optionally substituted heteroaryl; or alternatively Rₐ and R_{b} together with the nitrogen atom that they are connected to form a heterocycle comprising the nitrogen atom and 0-3 additional heteroatoms each selected from the group consisting of N, O, and S and optionally substituted by one or more alkyl, halogen, ORₐ, or oxo;

n₁ is an integer from 0-4; and

n₂ is an integer from 0-3;

with the proviso that the compound of Formula I is not H or

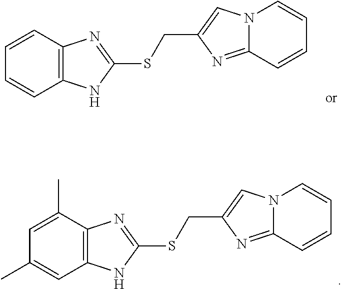 [SMILES: c1ccc2[nH]c(SCc3cn4ccccc4n3)nc2c1 ; Cc1cc(C)c2nc(SCc3cn4ccccc4n3)[nH]c2c1]

In some embodiments, the alkyl, cycloalkyl, heterocycle, aryl, and heteroaryl substituents described herein are each optionally and independently substituted by 1-4 substituents, each independently selected from the group consisting of alkyl, halogen, ORₐ, and oxo.

In some embodiments, n₁ is 0, 1, 2, or 3. In some embodiments, n₁ is 0. In other embodiments, n₁ is 1. In still other embodiments, n₁ is 2. In still other embodiments, n₁ is 3.

In some embodiments, at least one occurrence of R₁ is H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}. In some embodiments, at least one occurrence of R₁ is H, alkyl, or cycloalkyl. In other embodiments, at least one occurrence of R₁ is H. In still other embodiments, at least one occurrence of R₁ is alkyl, such as Me, Et, propyl, isopropyl, n-butyl, iso-butyl, or sec-butyl. In other embodiments, at least one occurrence of R₁ is cycloalkyl, such as cyclopropyl, cyclobutyl, cyclopentyl, or cyclohexyl.

In some embodiments, at least one occurrence of R₁ is halogen such as F, Cl, Br, or I. In some specific embodiments, at least one occurrence of R₁ is F, Cl, or Br. In some specific embodiments, at least one occurrence of R₁ is F or C₁. In some specific embodiments, at least one occurrence of R₁ is C₁.

In some embodiments, at least one occurrence of R₁ is halogenated alkyl. Non-limiting examples of halogenated alkyl include CF₃, CF₂H, CH₂CF₂H, CH₂CF₃, and CF₂CF₃.

In other embodiments, at least one occurrence of R₁ is ORₐ, CN, NRₐR_{b}, NO₂, (C═O)OR_{b}, NRₐ(C═O)R_{b}, or CONRₐR_{b}. In some specific embodiments, at least one occurrence of R₁ is ORₐ, such as OH, OMe, OEt, OPr, O-iso-Pr, OBu, O-tert-Bu, or O-sec-Bu. In other embodiments, at least one occurrence of R₁ is NRₐR_{b}, such as NH₂, NHMe, NMe₂, NHEt, NMeEt, NEt₂, NHPr, NMePr, NEtPr, N(Pr)₂, NH(iso-Pr), NMe(iso-Pr), NEt(iso-Pr), or N(iso-Pr)₂.

In still other embodiments, at least one occurrence of R₁ is CN or NO₂. In some specific embodiments, at least one occurrence of R₁ is NO₂. In still other embodiments, at least one occurrence of R₁ is (C═O)OR_{b}, NRₐ(C═O)R_{b}, or (C═O)NRₐR_{b}. In some specific embodiments, at least one occurrence of R₁ is COOH, COOMe, COOEt, NHAc, NMeAc, CONH₂, CONHMe, CONMe₂, CONHEt, CONMeEt, or CONEt₂.

In other embodiments, two R₁ groups and the carbon atoms they are connected to taken together form a 4-7 membered carbocycle or heterocycle optionally substituted by one or more alkyl, halogen, ORₐ, or oxo.

In some embodiments, R₂ is H or alkyl. In some embodiments, R₂ is H. In some embodiments, R₂ is alkyl, such as Me, Et, propyl, isopropyl, n-butyl, iso-butyl, or sec-butyl. In other embodiments, R₂ is cycloalkyl, such as cyclopropyl, cyclobutyl, cyclopentyl, or cyclohexyl.

In some embodiments, R₂ is heteroalkyl. In some specific embodiments, R₂ is alkyl ethers, secondary and tertiary alkyl amines, or alkyl sulfides, such as —CH₂-OEt, —CH₂—CH₂—OPr, —CH₂-SEt, —CH₂—CH₂—SPr, —CH₂—NHMe, or —CH₂—CH₂-NEtMe. In some embodiments, R₂ is cycloheteroalkyl. Non-limiting examples of cycloheteroalkyl include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane.

In some embodiments, the structural moiety

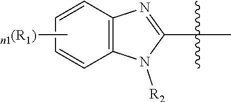

has the structure of

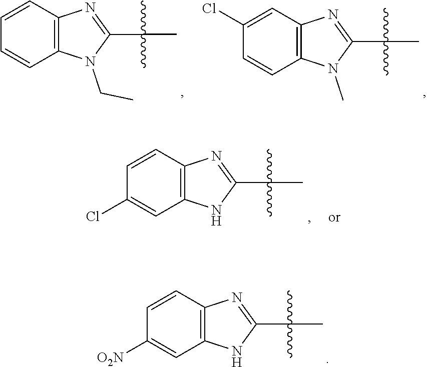 [SMILES: *c1nc2ccccc2n1CC ; *c1nc2cc(Cl)ccc2n1C ; *c1nc2ccc(Cl)cc2[nH]1 ; *c1nc2ccc([N+](=O)[O-])cc2[nH]1]

In some embodiments, -A-B- is —S—CR₄R₅—. In other embodiments, -A-B- is —CR₄R₅—S—. In some specific embodiments, R₄ and R₅ are each independently H or alkyl. In any of the embodiments described herein, —CR₄R₅— may be CH₂, CHCH₃, or C(CH₃)₂. In some specific embodiments herein, —CR₄R₅— is CH₂. In some embodiments, -A-B- is —S—CH₂—. In other embodiments, -A-B- is —CH₂—S—.

In other embodiments, at least one of R₄ and R₅ is cycloalkyl. Non-limiting examples of cycloalkyl include cyclopropyl, cyclobutyl, cyclopentyl, and cyclohexyl. In still other embodiments, R₄, R₅ and the carbon atom they are connected to taken together form a 3-7 membered carbocycle or heterocycle optionally substituted by one or more alkyl, halogen, ORₐ, or oxo.

In some embodiments, X is N. In other embodiments, X is CR₃. In some embodiments, Y is N. In other embodiments, Y is CR₃. In still other embodiments, X and Y are both N. In still other embodiments, X and Y are both CR₃. In some embodiments, the structural moiety

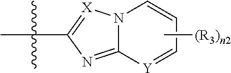

has a structure selected from the group consisting of

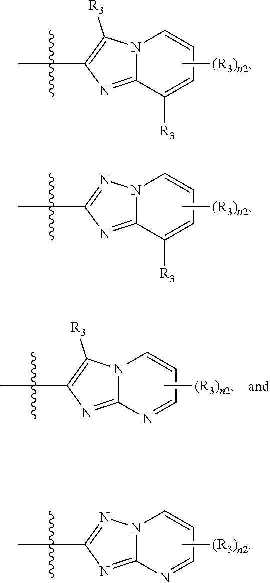

In some embodiments, n₂ is 0, 1, 2, or 3. In some embodiments, n₂ is 0. In other embodiments, n₂ is 1. In still other embodiments, n₂ is 2. In still other embodiments, n₂ is 3.

In some embodiments, at least one occurrence of R₃ is H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}. In some embodiments, at least one occurrence of R₃ is H, alkyl, or cycloalkyl. In some embodiments, at least one occurrence of R₃ is H. In some embodiments, each occurrence of R₃ is H. In other embodiments, at least one occurrence of R₃ is alkyl, such as Me, Et, propyl, isopropyl, n-butyl, iso-butyl, or sec-butyl. In other embodiments, at least one occurrence of R₃ is cycloalkyl, such as cyclopropyl, cyclobutyl, cyclopentyl, or cyclohexyl.

In some embodiments, at least one occurrence of R₃ is halogen such as F, Cl, Br, or I. In some specific embodiments, at least one occurrence of R₃ is F, Cl, or Br. In some specific embodiments, at least one occurrence of R₃ is F or C₁. In some specific embodiments, at least one occurrence of R₃ is H, F, Cl, Br, or Me.

In some embodiments, at least one occurrence of R₃ is halogenated alkyl. Non-limiting examples of halogenated alkyl include CF₃, CF₂H, CH₂CF₃, CH₂CF₂H, or CF₂CF₃.

In other embodiments, at least one occurrence of R₃ is ORₐ, CN, NRₐR_{b}, NO₂, (C═O)OR_{b}, NRₐ(C═O)R_{b}, or (C═O)NRₐR_{b}. In some specific embodiments, at least one occurrence of R₃ is ORₐ, such as OH, OMe, OEt, OPr, O-iso-Pr, OBu, O-tert-Bu, or O-sec-Bu. In other embodiments, at least one occurrence of R₃ is NRₐR_{b}, such as NH₂, NHMe, NMe₂, NHEt, NMeEt, NEt₂, NHPr, NMePr, NEtPr, NPr₂, NH(iso-Pr), NMe(iso-Pr), NEt(iso-Pr), or N(iso-Pr)₂.

In still other embodiments, at least one occurrence of R₃ is (C═O)OR_{b}, NRₐ(C═O)R_{b}, or (C═O)NRₐR_{b}. In some specific embodiments, at least one occurrence of R₃ is COOH, COOMe, COOEt, NHAc, NMeAc, CONH₂, CONHMe, CONMe₂, CONHEt, CONMeEt, or CONEt₂.

In other embodiments, two R₃ groups and the carbon atoms they are connected to taken together form a 4-7 membered carbocycle or heterocycle optionally substituted by one or more alkyl, halogen, ORₐ, or oxo.

In some embodiments, at least one of Rₐ and R_{b} is H, alkyl, or cycloalkyl. In some specific embodiments, at least one of Rₐ and R_{b} is H, Me, Et, propyl, isopropyl, butyl, sect-butyl, tert-butyl, cyclopropyl, cyclobutyl, cyclopentyl, or cyclohexyl.

In other embodiments, at least one of Rₐ and R_{b} is optionally substituted saturated heterocycle, optionally substituted aryl, or optionally substituted heteroaryl. In some specific embodiments, at least one of Rₐ and R_{b} is a carbocycle or heterocycle selected from the group consisting of

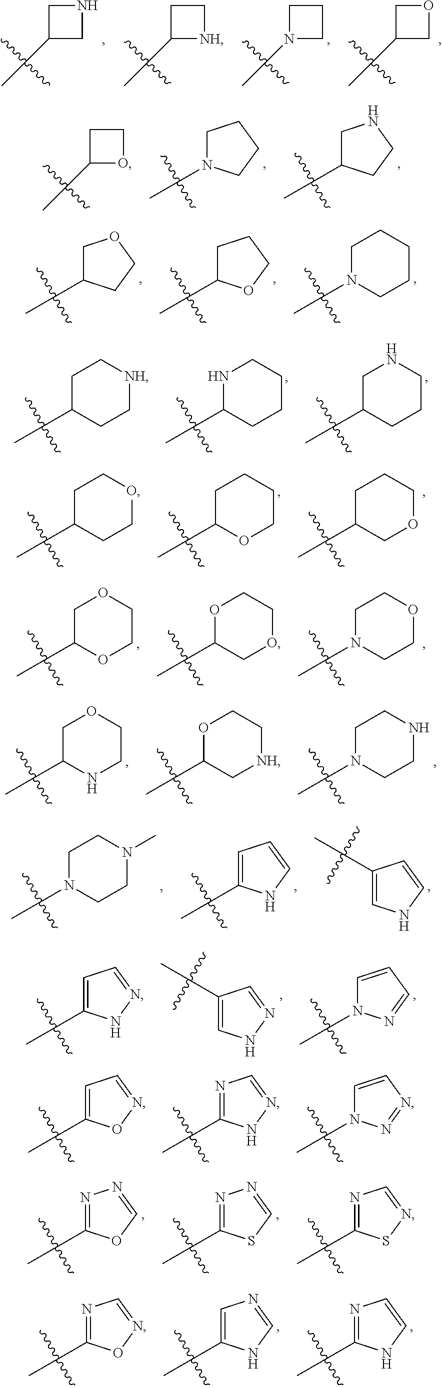

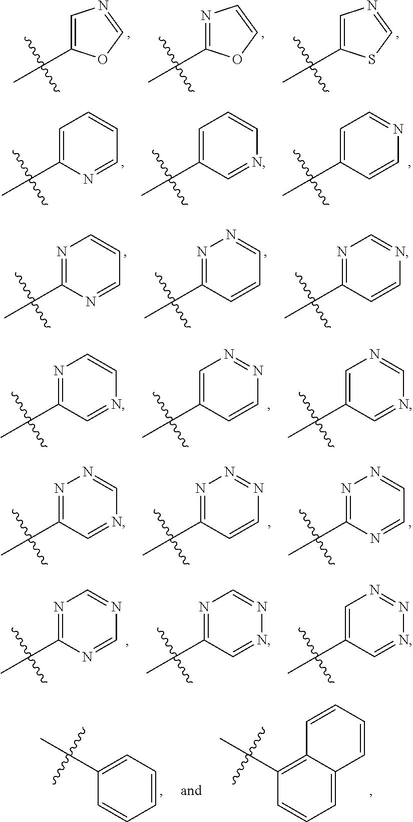

wherein the carbocycle or heterocycle is optionally substituted by one or more alkyl, ORₐ, or oxo where valence permits.

In other embodiments, Rₐ and R_{b} together with the nitrogen atom that they are connected to form an optionally substituted heterocycle comprising the nitrogen atom and 0-3 additional heteroatoms each selected from the group consisting of N, O, and S, wherein the heterocycle is optionally substituted by one or more alkyl, ORₐ, or oxo where valence permits.

In some embodiments, the structural moiety

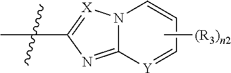

has the structure of

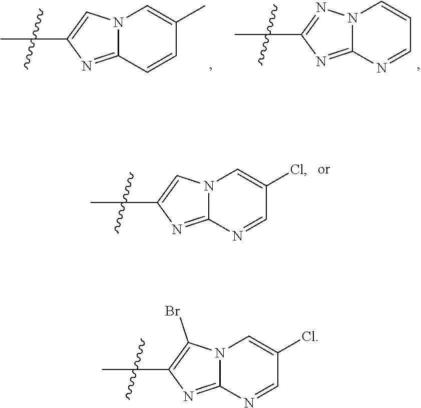 [SMILES: *c1cn2cc(C)ccc2n1 ; *c1nc2ncccn2n1 ; *c1cn2cc(Cl)cnc2n1 ; *c1nc2ncc(Cl)cn2c1Br]

In some embodiments, the compound of Formula I is selected from a group consisting of:

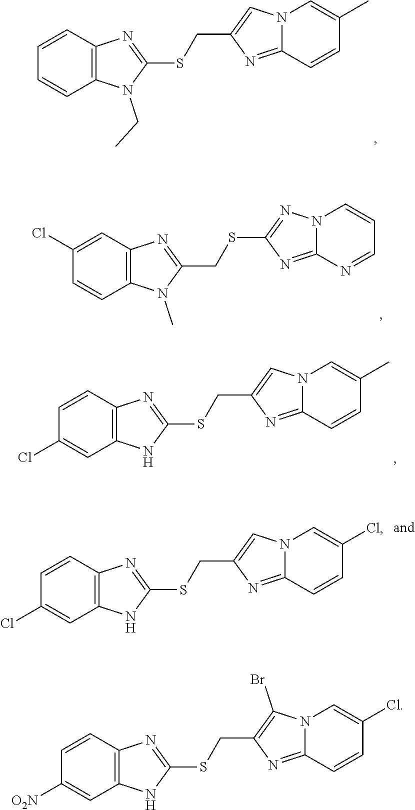 [SMILES: CCn1c(SCc2cn3cc(C)ccc3n2)nc2ccccc21 ; Cn1c(CSc2nc3ncccn3n2)nc2cc(Cl)ccc21 ; Cc1ccc2nc(CSc3nc4ccc(Cl)cc4[nH]3)cn2c1 ; Clc1ccc2nc(SCc3cn4cc(Cl)ccc4n3)[nH]c2c1 ; O=[N+]([O-])c1ccc2nc(SCc3nc4ccc(Cl)cn4c3Br)[nH]c2c1]

### Abbreviations

ACN Acetonitrile
EA Ethyl acetate
DMF Dimethyl formamide
DCM Dichloromethane
THF Tetrahydrofuran
DIPEA Diisopropylethylamine
NBS N-bromosuccinimide
TFA Trifluoroacetamide
MTBE Methyl tert-butyl ether
PE Petroleum ether

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). For example, the following reactions are illustrations but not limitations of the preparation of some of the starting materials and compounds disclosed herein.

Schemes 1-2 below describe synthetic routes which may be used for the synthesis of compounds of the present invention, e.g., compounds having a structure of Formula I and/or precursors thereof. Various modifications to these methods may be envisioned by those skilled in the art to achieve similar results to those given below. The general synthetic route described in Schemes 1-2 and examples described in the Example section below illustrate methods used for the preparation of the compounds described herein.

As shown in Scheme 1, compound Ia (i.e., compound of Formula I where -A-B- is —S—CR₄R₅—) can be prepared from compound II.

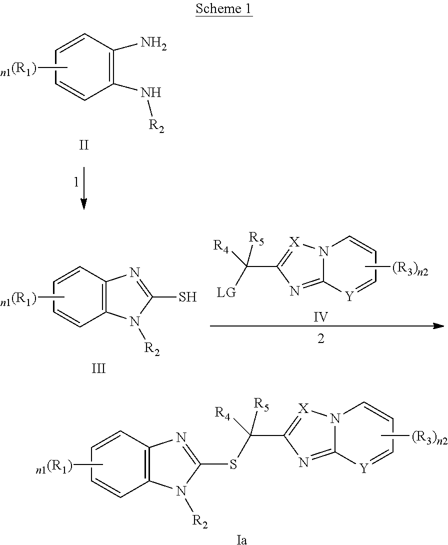

Compounds II and IV can be prepared by any method known in the art. As shown in Scheme 1, LG refers to a leaving group. As is known in the art, a leaving group refers to a substituent capable of being replaced by a nucleophile in a nucleophilic substitution reaction. Non-limiting examples of leaving groups include O(C═O)alkyl, O(C═O)Oalkyl, halides such as Cl⁻, Br⁻, and I⁻, and sulfonate esters such as tosylate (TsO⁻) or mesylates. Other substituents are defined herein. As shown in Scheme 1, step 1, compound II can be converted to thiobenzimidazole III through condensation reaction using a reagent such as CS₂ or 1-(imidazole-1-carbothioyl)imidazole. Other suitable reagents known in the art are contemplated. Compound III then undergoes a nucleophilic substitution reaction with compound IV (step 2) to afford the final compound, compound Ia. A base may be used in step 2. Non-limiting examples of the base include triethylamine, DIPEA, pyridine, and K₂CO₃.

As shown in Scheme 2, compound Ib (i.e., compound of Formula I where -A-B- is —CR₄R₅—S—) can be prepared from compound II.

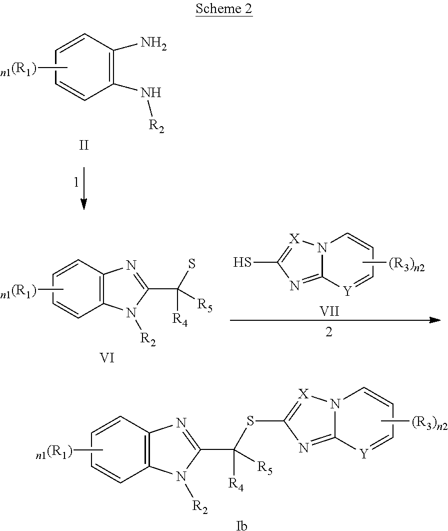

Compounds II and VII can be prepared by any method known in the art. As shown in Scheme 2, LG refers to a leaving group defined above. As shown in Scheme 2, step 1, compound II can be converted to benzimidazole VI through condensation reaction using a reagent such as sodium 2-chloroacetate (when LG is Cl). Other suitable reagents known in the art are contemplated. Compound VI then undergoes a nucleophilic substitution reaction with compound VII (step 2) to afford the final compound, compound Ib. A base may be used in step 2. Non-limiting examples of the base include triethylamine, DIPEA, pyridine, and K₂CO₃.

The reactions described in Schemes 1-2 can be carried out in a suitable solvent. Suitable solvents include, but are not limited to, acetonitrile, methanol, ethanol, dichloromethane, DMF, THF, MTBE, or toluene. The reactions described in Schemes 1-2 may be conducted under inert atmosphere, e.g., under nitrogen or argon, or the reaction may be carried out in a sealed tube. The reaction mixture may be heated in a microwave or heated to an elevated temperature using an oil bath. Suitable elevated temperatures include, but are not limited to, 40, 50, 60, 80, 90, 100, 110, 120° C., or higher or the refluxing/boiling temperature of the solvent used. The reaction mixture may alternatively be cooled in a cold bath at a temperature lower than room temperature, e.g., 0, −10, −20, −30, −40, −50, −78, and −90° C. The reaction may be worked up by removing the solvent or partitioning the organic solvent phase with one or more aqueous phases each optionally containing NaCl, NaHCO₃, or NH₄Cl. The solvent in the organic phase can be removed by reduced vacuum evaporation and the resulting residue may be purified using crystallization, a silica gel column or HPLC.

### Pharmaceutical Compositions

This invention also provides a pharmaceutical composition comprising at least one of the compounds as described herein or a pharmaceutically acceptable salt or solvate thereof, and a pharmaceutically acceptable carrier.

In yet another aspect, the present invention provides a pharmaceutical composition comprising at least one compound selected from the group consisting of compounds of Formula I as described herein and a pharmaceutically acceptable carrier or diluent.

In certain embodiments, the composition is in the form of a hydrate, solvate, or pharmaceutically acceptable salt. The composition can be administered to the subject by any suitable route of administration, including, without limitation, oral and parenteral.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is “acceptable” in the sense of being compatible with the other ingredients of the formulation and not injurious to the patient. Some examples of materials which can serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically acceptable salts. The term “pharmaceutically acceptable salt”, in this respect, refers to the relatively non-toxic, inorganic, and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al., (1977) “Pharmaceutical Salts”, J. Pharm. Sci. 66:1-19.)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic, and organic base addition salts of compounds of the present invention. These salts can likewise be prepared in situ during the final isolation and purification of the compounds, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine, and the like. (See, e.g., Berge et al., supra.)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules, and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active, or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required.

The ointments, pastes, creams, and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of this invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as propane and butane.

Transdermal patches have the added advantage of providing controlled delivery of a compound of the present invention to the body. Such dosage forms can be made by dissolving, or dispersing the pharmaceutical agents in the proper medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals to humans and animals, they can be givenper se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic agent). Non-limiting examples of another therapeutic agent including biological and small molecule anticancer agent, immunomodulator, immunosuppressant, anti-inflammatory agent, anti-arthritis agent, corticosteroid, antidiarrheal agent, anticoagulation agent, agent treating neurodegenerative diseases, and antithrombotic agent.

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating an amyloid-related disease in a mammalian species in need thereof, the method comprising administering to the mammalian species a therapeutically effective amount of at least one compound selected from the group consisting of compounds of Formula I, or a pharmaceutically acceptable salt thereof. In humans, amyloid formation has been linked to a plurality of diseases. As used herein, amyloid-related disease refers to a disease or condition linked to or caused by the formation of amyloid.

In some embodiments, the amyloid-related disease is selected from the group consisting of Alzheimer's disease, mild cognitive impairment, senile dementia, Down syndrome, cerebral amyloid angiopathy, inclusion body myositis, hereditary cerebral hemorrhage with amyloidosis (Dutch type), the Guam Parkinson-Dementia complex, macular degeneration, fronto-temporal dementia, Parkinson's disease, dementia with Lewy bodies, cerebrovascular type dementia, Pick's disease, Huntington's disease, dentatorubral pallidoluysian atrophy, spinocerebellar ataxia (SCA, types 1, 2, 3, 6, and 7), spinal and bulbar muscular atrophy, Creutzfeldt-Jakob disease, bovine spongiform encephalopathy in cows, scrapie in sheep, kuru, Gerstmann-Straussler-Scheinker disease, fatal familial insomnia, amyotrophic lateral sclerosis, familial British dementia, familial Danish dementia, hereditary cerebral hemorrhage with amyloidosis (HCHW A, Icelandic type), type II diabetes, dialysis-related amyloidosis, prostatic amyloid, primary systemic amyloidosis, systemic AL amyloidosis, nodular AL amyloidosis, myeloma associated amyloidosis, systemic (reactive) AA amyloidosis, secondary systemic amyloidosis, chronic inflammatory disease, familial Mediterranean fever, senile systemic amyloidosis, familial amyloid polyneuropathy, familial cardiac amyloid, familial visceral amyloidosis, hereditary non-neuropathic systemic amyloidosis, Finnish hereditary systemic amyloidosis, fibrinogen α-chain amyloidosis, insulin-related amyloidosis, medullary carcinoma of the thyroid, isolated atrial amyloidosis, cataract, progressive supranuclear palsy, multiple sclerosis, HIV-related dementia, senile cardiac amyloidosis, endocrine tumors, neuronal degradation, cortical visual deficits, glaucoma, ocular amyloidosis, primary retinal degeneration, optic nerve drusen, optic neuropathy, optic neuritis, lattice dystrophy, and a combination thereof.

In some embodiments, the mammalian species is human. In some embodiments, the macular degeneration is age-related macular degeneration.

In some embodiments, the amyloid-related disease is a neurodegenerative disorder. In some embodiments, the neurodegenerative disorder is selected from the group consisting of Alzheimer's disease, Parkinson's disease, dementia with Lewy bodies, Huntington's disease, cerebrovascular type dementia, Down syndrome, hereditary cerebral hemorrhage with amyloidosis (Dutch type), the Guam Parkinson-Dementia complex, mild cognitive impairment, Pick's disease, Creutzfeldt-Jakob disease, amyotrophic lateral sclerosis, and a combination thereof.

In some embodiments, the amyloid-related disease is an ocular disease associated with a β-amyloid-related pathological abnormality or change in the tissue of the visual system. The autoimmune disease is multiple sclerosis. In some embodiments, the ocular disease is selected from the group consisting of neuronal degradation, cortical visual deficits, glaucoma, cataract due to β-amyloid deposition, ocular amyloidosis, primary retinal degeneration, macular degeneration, optic nerve drusen, optic neuropathy, optic neuritis, lattice dystrophy, and a combination thereof.

In another aspect, a method of retaining or increasing cognitive memory capacity in a mammalian species (e.g., a human) suffering from memory impairment is described, including administering to the mammalian species a therapeutically effective amount of at least one compound of Formula I a pharmaceutically acceptable salt thereof. In yet another aspect, a method of reducing the β-amyloid plaque load, inhibiting the formation of β-amyloid plaques, and/or retarding the increase of amyloid load in the brain in a mammalian species (e.g., a human) in need thereof is described, comprising administering to the mammalian species a therapeutically effective amount of at least one compound of Formula I or a pharmaceutically acceptable salt thereof.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants, and even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds, in whose preparation excipients and additives and/or auxiliaries such as disintegrants, binders, coating agents, swelling agents, lubricants, flavorings, sweeteners, or solubilizers are customarily used as described above. The pharmaceutical compositions are suitable for use in a variety of drug delivery systems. For a brief review of present methods for drug delivery, see Langer R., Science, 249:1527-33, 1990, which is incorporated herein by reference.

In some embodiments, the concentration of compounds included in compositions used in the methods of the invention can range from about 1 nM to about 100 μM. In some embodiments, effective doses range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, depending on activity of the compound, manner of administration, purpose of the administration (i.e., prophylactic or therapeutic), nature, and severity of the disorder, age, and body weight of the patient, different doses may be necessary. The administration of a given dose can be carried out both by single administration in the form of an individual dose unit or by several smaller dose units. Repeated and multiple administration of doses at specific intervals of days, weeks, or months apart are also contemplated by the invention.

The compositions can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium, or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for administrations (e.g., subcutaneous, intramuscular, intraperitoneal, and intravenous administrations) can be found in Remington's Pharmaceutical Sciences, Mack Publishing Company, Easton, Pa.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters, and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

### Assays for Amyloid Inhibition

In some embodiments, the compounds as described herein are evaluated for their ability to inhibit amyloids.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

### Example 1. 6-chloro-1H-1,3-benzodiazole-2-thiol

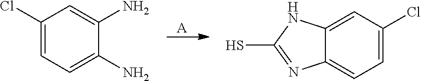

Step A: to a solution of 4-chlorobenzene-1,2-diamine (1.00 g, 7.01 mmol) in EtOH (8 mL) were added NaOH (0.30 g, 8.07 mmol), H₂O (1 mL), and CS₂ (0.6 g, 8.07 mmol) at room temperature. The resulting mixture was stirred at 80° C. for 3 h. After cooling to room temperature, the mixture was poured into a mixture of water (50 mL) and AcOH (5 mL) and stirred for an additional 2 h at room temperature. The solid was precipitated and filtered. The filter cake was washed with water (2×10 mL) and dried in a vacuum oven to afford 6-chloro-1H-1,3-benzodiazole-2-thiol as a grey solid (1.05 g, 81%): LCMS (ESI) calc'd for C₇H₅ClN₂S [M+H]⁺: 185, 187 (3:1), found 185, 187 (3:1); ¹H NMR (400 MHz, DMSO-d₆) δ 12.67 (s, 2H), 7.24-6.99 (m, 3H).

### Example 2. 6-chloro-2-(chloromethyl)imidazo[1,2-a]pyridine

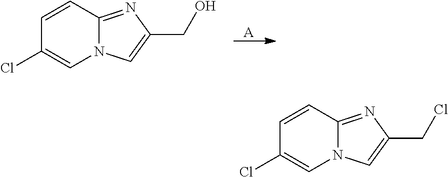

Step A: to a solution of [6-chloroimidazo[1,2-a]pyridin-2-yl]methanol (0.10 g, 0.55 mmol) in DCM (1 mL) was added SOCl₂ (65 mg, 0.55 mmol) dropwise at room temperature under nitrogen atmosphere. After stirring for additional 2 h, the resulting solution was concentrated under vacuum to afford 6-chloro-2-(chloromethyl)imidazo[1,2-a]pyridine (0.10 g, 90%) as an off-white solid: LCMS (ESI) calc'd for C₈H₆Cl₂N₂ [M+H]⁺: 201, 203 (3:2), found 201, 203 (3:2); ¹H NMR (400 MHz, DMSO-d₆) δ 9.15 (d, J=2.0, 1H), 8.26 (s, 1H), 7.91-7.78 (m, 2H), 5.04 (s, 2H).

### Example 3. 6-nitro-1H-1,3-benzodiazole-2-thiol

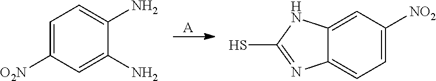

Step A: to a solution of 4-nitrobenzene-1,2-diamine (1.00 g, 6.53 mmol) in EtOH (10 mL) were added NaOH (0.30 g, 7.51 mmol), H₂O (1 mL), and CS₂ (5.00 g, 65.67 mmol) at room temperature. The reaction mixture was stirred at 80° C. for 16 h. After cooling to room temperature, the resulting solution was concentrated under vacuum and the residue was triturated with water (50 mL, plus 5 mL AcOH). The solid was precipitated and filtered. The filter cake was washed with water (2×10 mL) and dried in a vacuum oven to afford 6-nitro-1H-1,3-benzodiazole-2-thiol as a yellow solid (1.10 g, 86%): LCMS (ESI) calc'd for C₇H₅N₃O₂S [M+H]⁺: 196, found 196; ¹H NMR (400 MHz, DMSO-d₆) δ 12.49 (s, 2H), 8.07 (d, J=8.8 Hz 1H), 7.87 (s, 1H), 7.29 (d, J=8.8 Hz, 1H).

### Example 4. 3-bromo-6-chloro-2-(chloromethyl)imidazo[1,2-a]pyridine

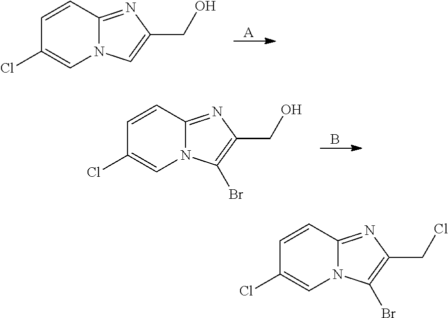

Step A: to a solution of [6-chloroimidazo[1,2-a]pyridin-2-yl]methanol (1.00 g, 5.48 mmol) in MeCN (20 mL) was added dropwise a solution of NBS (1.00 g, 5.48 mmol) in MeCN (5 mL) over 5 min at 0° C. The resulting mixture was allowed to warm to room temperature and stirred at room temperature for 2 h under nitrogen atmosphere. The reaction mixture was concentrated under vacuum and the residue was triturated with a co-solvent (10 mL, PE:EA=1:1). The solid was collected by filtration and dried in a vacuum oven to afford [3-bromo-6-chloroimidazo[1,2-a]pyridin-2-yl]methanol as an off-white solid (1.30 g, 90%): LCMS (ESI) calc'd for C₈H₆BrClN₂O [M+H]⁺: 261, 263, 265 (2:3:1), found 261, 263, 265 (2:3:1); ¹H NMR (400 MHz, CD₃OD) δ 8.46 (d, J=2.0 Hz, 1H), 7.60 (d, J=9.6 Hz, 1H), 7.43 (dd, J=9.6, 2.0 Hz, 1H), 4.74 (s, 2H).

Step B: to a solution of [3-bromo-6-chloroimidazo[1,2-a]pyridin-2-yl]methanol (0.50 g, 1.91 mmol) in DCM (5 mL) was added SOCl₂ (0.15 mL, 2.07 mmol) at 0° C. The resulting solution was allowed to warm to room temperature and stirred for additional 2 h. Then the resulting solution was concentrated under vacuum to afford 3-bromo-6-chloro-2-(chloromethyl)imidazo[1,2-a]pyridine as an off-white solid (0.50 g, 93%): LCMS (ESI) calc'd for C₈H₅BrCl₂N₂ [M+H]⁺: 279, 281, 283 (1:2:1) found 279, 281, 283 (1:2:1); ¹H NMR (400 MHz, DMSO-d₆) δ 8.7 (d, J=2.0 Hz, 1H), 7.72 (d, J=9.6 Hz, 1H), 7.50 (dd, J=9.6, 2.0 Hz, 1H), 4.86 (s, 2H).

### Example 5. 5-chloro-2-(chloromethyl)-1-methyl-1,3-benzodiazole

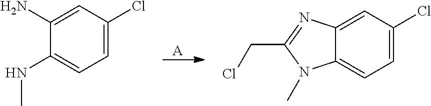

Step A: a mixture of 4-chloro-N¹-methylbenzene-1,2-diamine (0.20 g, 1.28 mmol) and sodium 2-chloroacetate (0.24 g, 2.55 mmol) in aq. HCl (6 N, 3.00 mL) was stirred for 16 h at 80° C. After cooling to room temperature, the resulting solution was neutralized with saturated aqueous NaHCO₃. The aqueous phase was extracted with EA (3×20 mL). The combined organic layers were washed with brine (3×10 mL) and dried over anhydrous Na₂SO₄. After filtration, the filtrate was concentrated under vacuum. The residue was purified by silica gel column chromatography, eluted with PE/EA (5:1) to afford 5-chloro-2-(chloromethyl)-1-methyl-1,3-benzodiazole as a light yellow solid (0.19 g, 69%): LCMS (ESI) calc'd for C₉H₈Cl₂N₂ [M+H]⁺: 215, 217 (3:2), found 215, 217 (3:2); ¹H NMR (400 MHz, CD₃OD) δ 7.76 (s, 1H), 7.56 (d, J=8.4 Hz, 1H), 7.35 (d, J=8.4, 1H), 4.86 (s, 2H), 3.94 (s, 3H).

### Example 6. 2-(chloromethyl)-6-methylimidazo[1,2-a]pyridine

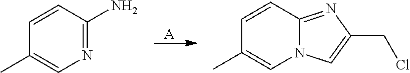

Step A: a mixture of 2-amino-5-methylpyridine (1.00 g, 9.25 mmol) and 1,3-dichloroacetone (1.76 g, 13.87 mmol) in ACN (15 mL) was stirred for 16 h at 80° C. After cooling down to room temperature, the reaction was quenched with water (30 mL) and extracted with EA (3×20 mL). The combined organic layers were washed with brine (3×10 mL) and dried over anhydrous Na₂SO₄. After filtration, the filtrate was concentrated under vacuum. The residue was purified by silica gel column chromatography, eluted with PE/EA (1:1) to afford 2-(chloromethyl)-6-methylimidazo[1,2-a]pyridine as a light yellow solid (0.40 g, 24%): LCMS (ESI) calc'd for C₉H₉ClN₂ [M+H]⁺: 181, 183 (3:1), found 181, 183 (3:1); ¹H NMR (400 MHz, CD₃OD) δ 8.20 (s, 1H), 7.80 (s, 1H), 7.42 (d, J=9.2 Hz, 1H), 7.23 (dd, J=9.2, 1H), 4.76 (s, 2H), 2.34 (s, 3H).

### Example 7. 1-ethyl-1,3-benzodiazole-2-thiol

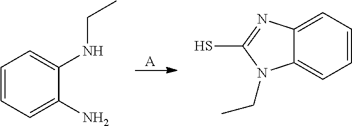

Step A: to a stirred mixture of N¹-ethylbenzene-1,2-diamine hydrochloride (0.20 g, 1.47 mmol) and Et₃N (0.45 g, 4.41 mmol) in THE (4 mL) was added 1-(imidazole-1-carbothioyl)imidazole (0.29 g, 1.62 mmol) in portions at room temperature under air atmosphere. The resulting mixture was stirred for 16 h at room temperature. The reaction was quenched with water (30 mL) and extracted with EA (3×15 mL). The combined organic layers were washed with brine (2×10 mL) and dried over anhydrous Na₂SO₄. After filtration, the filtrate was concentrated under vacuum. The residue was purified by silica gel column chromatography, eluted with PE/EA (5:1) to afford 1-ethyl-1,3-benzodiazole-2-thiol as a light yellow solid (0.16 g, 61%): LCMS (ESI) calc'd for C₉H₁₀N₂S [M+H]⁺: 179, found 179; ¹H NMR (400 MHz, CDCl₃) δ 10.67 (s, 1H), 7.27-7.19 (m, 4H), 4.39 (q, J=7.2 Hz, 2H), 1.45 (t, J=6.2 Hz, 3H).

### Example 8. Compound 1 (6-chloro-2-[([6-chloroimidazo[1,2-a]pyridin-2-yl]methyl)sulfanyl]-1H-1,3-benzodiazole)

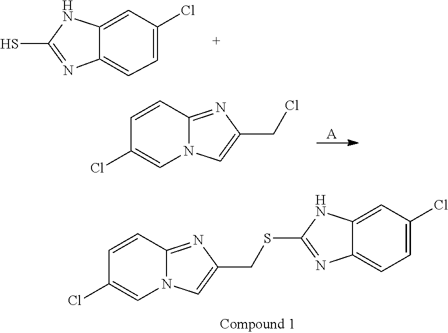

Step A: to a mixture of 6-chloro-2-(chloromethyl)imidazo[1,2-a]pyridine (0.10 g, 0.50 mmol) (Example 2) and 6-chloro-1H-1,3-benzodiazole-2-thiol (92 mg, 0.50 mmol) (Example 1) in DMF (2.0 mL) was added DIPEA (0.2 mL, 1.59 mmol) in one portion at room temperature. The resulting solution was stirred at 60° C. for 2 h. After cooling to room temperature, the resulting solution was diluted with water (30 mL) and extracted with EA (2×20 mL). The combined organic phases were washed with brine (3×5 mL), dried over anhydrous Na₂SO₄ and filtered. The filtrate was concentrated under vacuum. The residue was purified with Prep-HPLC with the following conditions: Column: Sunfire Prep C18 OBD Column, 10 μm, 19×250 mm; Mobile Phase A: water (plus 0.05% TFA), Mobile Phase B: ACN; Flow rate: 25 mL/min; Gradient: 20 B to 58 B in 6 min; Detector: UV 254 nm; Retention time: 5.8 min. The fractions containing the desired product were collected and concentrated under vacuum to afford Compound 1 (6-chloro-2-[([6-chloroimidazo[1,2-a]pyridin-2-yl]methyl)sulfanyl]-1H-1,3-benzodiazole trifluoroacetic acid) as a light brown semi-solid (105.8 mg, 60%): LCMS (ESI) calc'd for C₁₅H₁₀Cl₂N₄S [M+H]⁺: 349, 351 (3:2), found 349, 351 (3:2); ¹H NMR (400 MHz, DMSO-d₆) δ 9.02 (s, 1H), 8.10 (s, 1H), 7.83 (d, J=9.4 Hz, 1H), 7.72 (d, J=9.4 Hz, 1H), 7.57 (d, J=2.0 Hz, 1H), 7.49 (d, J=8.4 Hz, 1H), 7.19 (dd, J=8.6, 2.0 Hz, 1H); 4.77 (s, 2H); ¹⁹F NMR (376 MHz, CD₃OD) δ− 74.23.

### Example 9. Compound 2 (2-[([3-bromo-6-chloroimidazo[1,2-a]pyridin-2-yl]methyl)sulfanyl]-6-nitro-1H-1,3-benzodiazole)

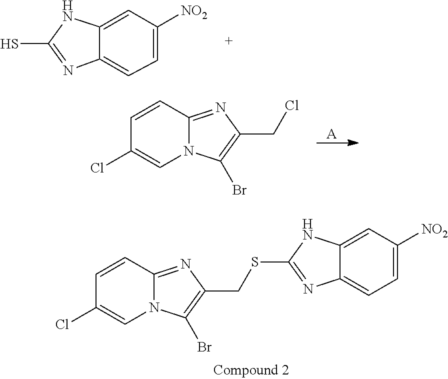

Step A: to a solution of 3-bromo-6-chloro-2-(chloromethyl)imidazo[1,2-a]pyridine (0.10 g, 0.36 mmol) (Example 4) and 6-nitro-1H-1,3-benzodiazole-2-thiol (69 mg, 0.36 mmol) (Example 3) in DMF (2 mL) was added DIPEA (0.12 g, 0.89 mmol) in one portion at room temperature. The resulting solution was stirred at 60° C. for 2 h. After cooling to room temperature, the resulting mixture was diluted with water (30 mL) and extracted with EA (2×20 mL). The combined organic phases were washed with brine (3×5 mL), dried over anhydrous Na₂SO₄ and filtered. The filtrate was concentrated under vacuum. The residue was purified with Prep-HPLC with the following conditions: Column: SunFire C18 OBD Prep Column, 100 Å, 5 μm, 19 mm×250 mm; Mobile Phase A: water (plus 0.05% TFA), Mobile Phase B: ACN; Flow rate: 25 mL/min; Gradient: 50 B to 90 B in 6.5 min; Detector: UV 254 nm; Retention time: 6 min. The fractions containing the desired product were collected and concentrated under vacuum to afford Compound 2 (2-[([3-bromo-6-chloroimidazo[1,2-a]pyridin-2-yl]methyl)sulfanyl]-6-nitro-1H-1,3-benzodiazole trifluoroacetic acid) as an orange solid (63.6 mg, 41%): LCMS (ESI) calc'd for C₁₅H₉BrClN₅O₂S [M+H]⁺: 438, 440, 442 (2:3:1) found 438, 440, 442 (2:3:1); ¹H NMR (400 MHz, DMSO-d₆) δ 13.41 (s, 1H), 8.50 (s, 1H), 8.35 (s, 1H), 8.08 (dd, J=9.0, 2.2 Hz, 1H), 7.71-7.60 (m, 2H), 7.43 (dd, J=9.6, 2.0 Hz, 1H), 4.77 (s, 2H); ¹⁹F NMR (376 MHz, CD₃OD) δ− 73.74.

### Example 10. Compound 3 (5-chloro-1-methyl-2-([[1,2,4]triazolo[1,5-a]pyrimidin-2-ylsulfanyl]methyl)-1,3-benzodiazole)

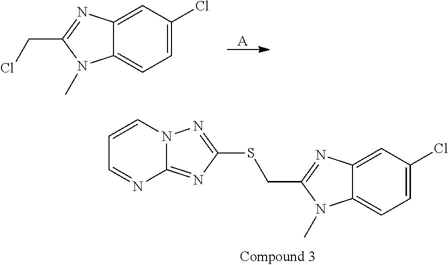

Step A: to a stirred solution of 5-chloro-2-(chloromethyl)-1-methyl-1,3-benzodiazole (0.10 g, 0.46 mmol) (Example 5) and [1,2,4]triazolo[1,5-a]pyrimidine-2-thiol (0.11 g, 0.70 mmol) in DMF (2 mL) was added K₂CO₃ (0.19 g, 1.39 mmol) in portions at room temperature. The resulting mixture was stirred for 16 h at 60° C. After cooling to room temperature, solids were precipitated and collected by filtration. The filter cake was washed with MeOH (10 mL), H₂O (4×10 mL) and dried in a vacuum oven to afford Compound 3 (5-chloro-1-methyl-2-([[1,2,4]triazolo[1,5-a]pyrimidin-2-ylsulfanyl]methyl)-1,3-benzodiazole) as a light yellow solid (82 mg, 53%): LCMS (ESI) calc'd for C₁₄H₁₁ClN₆S [M+H]⁺: 331, 333 (3:1), found 331, 333 (3:1); ¹H NMR (400 MHz, DMSO-d₆) δ 9.34 (dd, J=6.8, 2.0 Hz, 1H), 8.84 (dd, J=4.4, 2.0 Hz, 1H), 7.65 (d, J=2.0 Hz, 1H), 7.61 (d, J=8.6 Hz, 1H), 7.33 (dd, J=6.8, 4.4 Hz, 1H), 7.28 (dd, J=8.6, 2.0 Hz, 1H), 4.88 (s, 2H), 3.91 (s, 3H).

### Example 11. Compound 4 (1-ethyl-2-[([6-methylimidazo[1,2-a]pyridin-2-yl]methyl)sulfanyl]-1,3-benzodiazole)

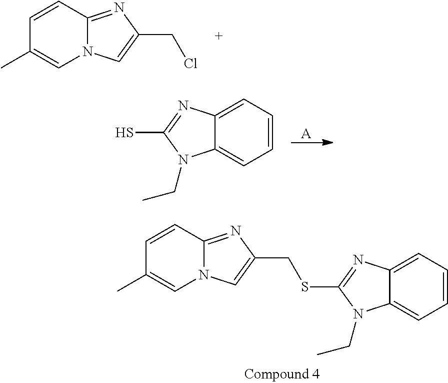

Step A: to a stirred solution of 1-ethyl-1,3-benzodiazole-2-thiol (0.15 g, 0.84 mmol) (Example 7) and 2-(chloromethyl)-6-methylimidazo[1,2-a]pyridine (0.23 g, 1.26 mmol) (Example 6) in DMF (3 mL) was added K₂CO₃ (0.35 g, 2.53 mmol) in portions at room temperature. The resulting mixture was stirred for 6 h at 60° C. under nitrogen atmosphere. After cooling down to room temperature, the reaction was diluted with water (30 mL) and extracted with EA (3×30 mL). The combined organic layers were washed with brine (2×10 mL) and dried over anhydrous Na₂SO₄. After filtration, the filtrate was concentrated under vacuum. The residue was purified by Prep-HPLC with the following conditions: Column: XBridge Shield RP18 OBD Column 30×150 mm, 5 m; Mobile Phase A: water (plus 0.05% TFA), Mobile Phase B: ACN; Flow rate: 60 mL/min; Gradient: 10% B to 30% B in 7 min; Detector: UV 254/220 nm; Retention time: 6.5 min. The fractions containing the desired product were collected and concentrated under vacuum to afford Compound 4 (1-ethyl-2-[([6-methylimidazo[1,2-a]pyridin-2-yl]methyl)sulfanyl]-1,3-benzodiazole trifluoroacetic acid) as a light yellow semi-solid (188 mg, 51%): LCMS (ESI) calc'd for C₁₈H₁₈N₄S [M+H]⁺: 323, found 323; ¹H NMR (400 MHz, CD₃OD) δ 8.51 (s, 1H), 8.09 (s, 1H), 7.88-7.77 (m, 2H), 7.74-7.65 (m, 1H), 7.63-7.54 (m, 1H), 7.43-7.31 (m, 2H), 4.84 (s, 2H), 4.36 (q, J=7.2 Hz, 2H), 2.46 (s, 3H), 1.41 (t, J=7.2 Hz, 3H); ¹⁹F NMR (376 MHz, CD₃OD) δ −77.49.

### Example 12. Evaluation of Amyloid Inhibition Activities

This assay is used to evaluate the disclosed compounds' activities for inhibiting amyloids.

### Thioflavin T (ThT) Beta-Amyloid (1-42) Aggregation Assay Protocol

### Reagents

SensoLyte Thioflavin T Beta-Amyloid (1-42) Aggregation Kit was used (AnaSpec Cat #AS-72214), which includes the following components:

Component A: Assay Buffer (Buffer components are not disclosed);

Component B: Beta-Amyloid (1-42), human, 0.25 mg×2, lyophilized;

Component C: 20 mM ThT solution; and

Component D: 20 mM Phenol Red (Control Inhibitor).

### Reaction Buffer

The following buffer was used: 0.005% Tween 20, Assay Buffer from Kit (Component A), and 1% DMSO (final including carryover from compounds).

### Reaction Condition (Final)

The final reaction conditions include 8 μM Beta-Amyloid (1-42) and 50 μM ThT dye.

### Assay Plate

Corning cat #3573, Non-Treated, Black 384-well plate was used as assay plate.

### Control Inhibitor

Morin (Sigma Cat #: M4008-2G) was used as control inhibitor.

### Reaction Procedure

The reaction procedure is as follows:
1. Prepare 2×ThT solution in Reaction Buffer; keep in the dark;
2. Deliver 10 μl/well of 2×ThT solution into the wells;
3. Prepare 2× Beta-Amyloid solution in Reaction Buffer in polypropylene tube, sonicate for 5 min; keep on ice until use;
4. Deliver compounds in DMSO into the ThT solution by Acoustic technology (Echo550; nanoliter range), no pre-incubation;
5. Immediately after compound addition, deliver 10 μL/well of Beta-Amyloid solution into the reaction well, buffer into “No peptide” wells instead;
6. Immediately start measuring fluorescence intensity in EnVision, set at 37° C., (Ex/Em=450/485 nm) as a time-course measurement every 5 min (30 sec shaking between each read) for 3 hours;
7. Analyze data by taking slope (signal/time) of linear portion (typically 5 to 45 min) of measurement (therefore time period taken for slope for analysis is different for each target); and
8. Slope is calculated by using Excel, and curve fits are performed using Prism software.

Table 1 provides a summary of the activities of certain selected compounds of Formula (I) for inhibiting amyloid.

TABLE 1
IC₅₀ (μM) values of certain exemplified compounds of Formula (I) for inhibiting amyloids.

| Compound | | |
|---|---|---|
| Number | Structure | IC₅₀ |
| 1 | 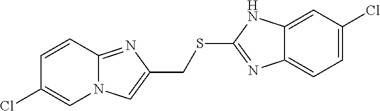 [SMILES: Clc1ccc2nc(SCc3cn4cc(Cl)ccc4n3)[nH]c2c1] | A |
| 2 | 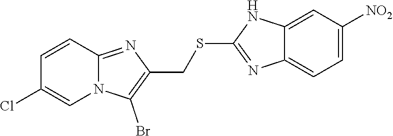 [SMILES: O=[N+]([O-])c1ccc2nc(SCc3nc4ccc(Cl)cn4c3Br)[nH]c2c1] | B |
| 3 | 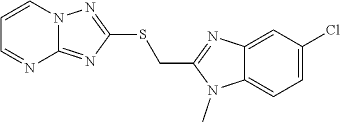 [SMILES: Cn1c(CSc2nc3ncccn3n2)nc2cc(Cl)ccc21] | B |
| 4 | 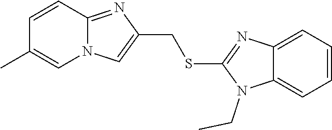 [SMILES: CCn1c(SCc2cn3cc(C)ccc3n2)nc2ccccc21] | A |
| 5 | 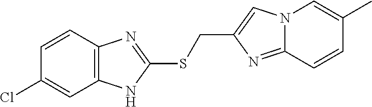 [SMILES: Cc1ccc2nc(CSc3nc4ccc(Cl)cc4[nH]3)cn2c1] | B |

A indicates that the tested compound has an IC₅₀ of less than 100 μM.
B indicates that the tested compound has an IC₅₀ of less than 200 μM.

1. A compound of Formula I or a pharmaceutically acceptable salt thereof, 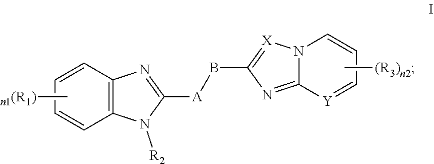
wherein
each occurrence of R₁ is independently H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}; or alternatively two R₁ groups and the carbon atoms they are connected to taken together form a 4-7 membered carbocycle or heterocycle optionally substituted by one or more alkyl, halogen, ORₐ, or oxo;
R₂ is H, alkyl, heteroalkyl, cycloalkyl, or cycloheteroalkyl;
-A-B- is —S—CR₄R₅— or —CR₄R₅—S—;
R₄ and R₅ are each independently H, alkyl, or cycloalkyl; or alternatively R₄, R₅ and the carbon atom they are connected to taken together form a 3-7 membered carbocycle or heterocycle optionally substituted by one or more alkyl, halogen, ORₐ, or oxo;
X is N or CR₃;
Y is N or CR₃;
each occurrence of R₃ is independently H, alkyl, halogenated alkyl, cycloalkyl, halogen, ORₐ, CN, NRₐR_{b}, NO₂, (C═O)OR_{b}, NRₐ(C═O)R_{b}, or CONRₐR_{b}; or alternatively two R₃ groups and the carbon atoms they are connected to taken together form a 4-7 membered carbocycle or heterocycle optionally substituted by one or more alkyl, halogen, ORₐ, or oxo;
each occurrence of Rₐ and R_{b} are independently H, alkyl, cycloalkyl, optionally substituted saturated heterocycle, optionally substituted aryl, or optionally substituted heteroaryl; or alternatively Rₐ and R_{b} together with the nitrogen atom that they are connected to form a heterocycle comprising the nitrogen atom and 0-3 additional heteroatoms each selected from the group consisting of N, O, and S and optionally substituted by one or more alkyl, halogen, ORₐ, or oxo;
n₁ is an integer from 0-4; and
n₂ is an integer from 0-3;
with the proviso that the compound of Formula I is not H or

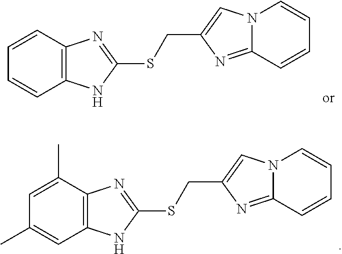 [SMILES: c1ccc2[nH]c(SCc3cn4ccccc4n3)nc2c1 ; Cc1cc(C)c2nc(SCc3cn4ccccc4n3)[nH]c2c1]

2. The compound of claim 1, wherein n₁ is 0, 1, or 2.

3. The compound of claim 1, wherein at least one occurrence of R₁ is halogen or NO₂.

4. The compound of claim 3, wherein at least one occurrence of R₁ is F, Cl, or NO₂.

5. The compound of claim 4, wherein at least one occurrence of R₁ is F or Cl.

6. The compound of claim 1, wherein at least one occurrence of R₁ is H, alkyl, halogenated alkyl, cycloalkyl, ORₐ, CN, or (C═O)OR_{b}.

7. The compound of claim 1, wherein at least one occurrence of R₁ is NRₐR_{b}, NRₐ(C═O)R_{b}, or CONRₐR_{b}.

8. The compound of claim 1, wherein R₂ is H, alkyl, or cycloalkyl.

9. The compound of claim 8, wherein R₂ is H, CH₃, or CH₂CH₃.

10. The compound of claim 1, wherein R₂ is heteroalkyl or cycloheteroalkyl.

11. The compound of claim 1, wherein -A-B- is —S—CR₄R₅—.

12. The compound of claim 1, wherein -A-B- is —CR₄R₅—S—.

13. The compound of claim 1, wherein at least one of R₄ and R₅ is H or alkyl.

14. The compound of claim 13, wherein CR₄R₅ is CH₂, CHCH₃, or C(CH₃)₂.

15. The compound of claim 1, wherein at least one of R₄ and R₅ is cycloalkyl.

16. The compound of claim 1, wherein X is N.

17. The compound of claim 1, wherein X is CR₃.

18. The compound of claim 1, wherein Y is N.

19. The compound of claim 1, wherein Y is CR₃.

20. The compound of claim 1, wherein X and Y are both N.

21. The compound of claim 1, wherein X and Y are both CR₃.

22. The compound of claim 1, wherein at least one occurrence of R₃ is H, alkyl, halogenated alkyl, or halogen.

23. The compound of claim 22, wherein at least one occurrence of R₃ is H, CH₃, CH₂CH₃, F, Cl, or Br.

24. The compound of claim 1, wherein at least one occurrence of R₃ is cycloalkyl, ORₐ, CN, (C═O)OR_{b}, or NO₂.

25. The compound of claim 1, wherein at least one occurrence of R₃ is NRₐR_{b}, NRₐ(C═O)R_{b}, or CONRₐR_{b}.

26. The compound of claim 1, wherein n₂ is 0, 1, or 2.

27. The compound of claim 1, wherein at least one of Rₐ and R_{b} is H, alkyl, or cycloalkyl.

28. The compound of claim 26, wherein at least one of Rₐ and R_{b} is H, CH₃, CH₂CH₃, propyl, isopropyl, cyclopropyl, or cyclobutyl.

29. The compound of claim 1, wherein at least one of Rₐ and R_{b} is optionally substituted saturated heterocycle, optionally substituted aryl, or optionally substituted heteroaryl.

30. The compound of claim 1, wherein Rₐ and R_{b} together with the nitrogen atom that they are connected to form an optionally substituted heterocycle comprising the nitrogen atom and 0-3 additional heteroatoms each selected from the group consisting of N, O, and S.

31. The compound of claim 1, wherein the structural moiety 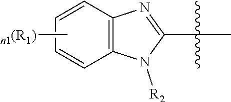
has the structure of

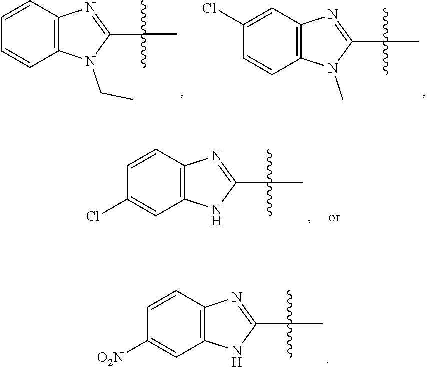 [SMILES: *c1nc2ccccc2n1CC ; *c1nc2cc(Cl)ccc2n1C ; *c1nc2ccc(Cl)cc2[nH]1 ; *c1nc2ccc([N+](=O)[O-])cc2[nH]1]

32. The compound of claim 1, wherein the structural moiety 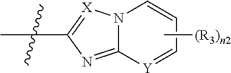
has the structure of

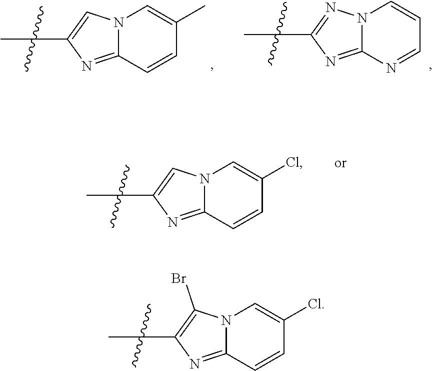 [SMILES: *c1cn2cc(C)ccc2n1 ; *c1nc2ncccn2n1 ; *c1cn2cc(Cl)ccc2n1 ; *c1nc2ccc(Cl)cn2c1Br]

33. The compound of claim 1 selected from the group consisting of

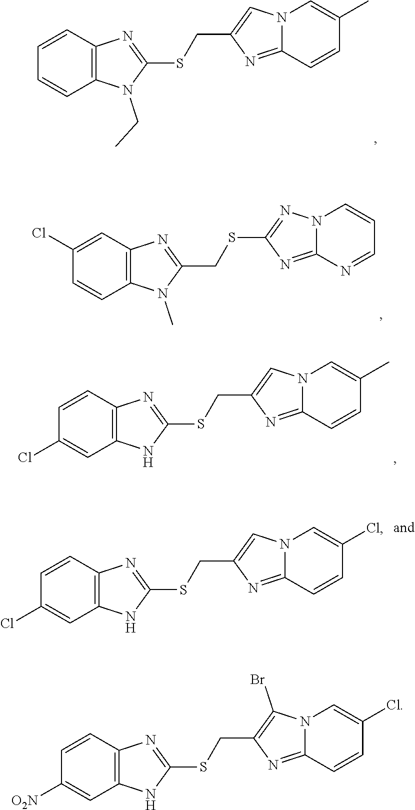 [SMILES: CCn1c(SCc2cn3cc(C)ccc3n2)nc2ccccc21 ; Cn1c(CSc2nc3ncccn3n2)nc2cc(Cl)ccc21 ; Cc1ccc2nc(CSc3nc4ccc(Cl)cc4[nH]3)cn2c1 ; Clc1ccc2nc(SCc3cn4cc(Cl)ccc4n3)[nH]c2c1 ; O=[N+]([O-])c1ccc2nc(SCc3nc4ccc(Cl)cn4c3Br)[nH]c2c1]

34. A pharmaceutical composition comprising at least one compound according to claim 1 or a pharmaceutically acceptable salt thereof and a pharmaceutically acceptable carrier or diluent.

35. A method of treating an amyloid-related disease in a mammalian species in need thereof, comprising administering to the mammalian species a therapeutically effective amount of at least one compound according to claim 1 or a pharmaceutically acceptable salt thereof.

36. The method of claim 35, wherein the amyloid-related disease is selected from the group consisting of Alzheimer's disease, mild cognitive impairment, senile dementia, Down syndrome, cerebral amyloid angiopathy, inclusion body myositis, hereditary cerebral hemorrhage with amyloidosis (Dutch type), the Guam Parkinson-Dementia complex, macular degeneration, fronto-temporal dementia, Parkinson's disease, dementia with Lewy bodies, cerebrovascular type dementia, Pick's disease, Huntington's disease, dentatorubral pallidoluysian atrophy, spinocerebellar ataxia (SCA, types 1, 2, 3, 6, and 7), spinal and bulbar muscular atrophy, Creutzfeldt-Jakob disease, bovine spongiform encephalopathy in cows, scrapie in sheep, kuru, Gerstmann-Straussler-Scheinker disease, fatal familial insomnia, amyotrophic lateral sclerosis, familial British dementia, familial Danish dementia, hereditary cerebral hemorrhage with amyloidosis (HCHW A, Icelandic type), type II diabetes, dialysis-related amyloidosis, prostatic amyloid, primary systemic amyloidosis, systemic AL amyloidosis, nodular AL amyloidosis, myeloma associated amyloidosis, systemic (reactive) AA amyloidosis, secondary systemic amyloidosis, chronic inflammatory disease, familial Mediterranean fever, senile systemic amyloidosis, familial amyloid polyneuropathy, familial cardiac amyloid, familial visceral amyloidosis, hereditary non-neuropathic systemic amyloidosis, Finnish hereditary systemic amyloidosis, fibrinogen α-chain amyloidosis, insulin-related amyloidosis, medullary carcinoma of the thyroid, isolated atrial amyloidosis, cataract, progressive supranuclear palsy, multiple sclerosis, HIV-related dementia, senile cardiac amyloidosis, endocrine tumors, neuronal degradation, cortical visual deficits, glaucoma, ocular amyloidosis, primary retinal degeneration, optic nerve drusen, optic neuropathy, optic neuritis, lattice dystrophy, and a combination thereof.

37. The method of claim 36, wherein the macular degeneration is age-related macular degeneration.

38. The method of claim 35, wherein the amyloid-related disease is a neurodegenerative disorder.

39. The method of claim 38, wherein the neurodegenerative disorder is selected from the group consisting of Alzheimer's disease, Parkinson's disease, dementia with Lewy bodies, Huntington's disease, cerebrovascular type dementia, Down syndrome, hereditary cerebral hemorrhage with amyloidosis (Dutch type), the Guam Parkinson-Dementia complex, mild cognitive impairment, Pick's disease, Creutzfeldt-Jakob disease, amyotrophic lateral sclerosis, and a combination thereof.

40. The method of claim 35, wherein the amyloid-related disease is an ocular disease associated with a β-amyloid-related pathological abnormality or change in the tissue of the visual system.

41. The method of claim 40, wherein the ocular disease is selected from the group consisting of cortical visual deficits, glaucoma, cataract due to β-amyloid deposition, ocular amyloidosis, primary retinal degeneration, macular degeneration, optic nerve drusen, optic neuropathy, optic neuritis, and lattice dystrophy.

42. The method of claim 35, wherein the mammalian species is human.

43. A method of retaining or increasing cognitive memory capacity in a mammalian species suffering from memory impairment, comprising administering to the mammalian species a therapeutically effective amount of at least one compound according to claim 1 or a pharmaceutically acceptable salt thereof.

44. The method of claim 43, wherein the mammalian species is human.

45. A method of reducing the β-amyloid plaque load, inhibiting the formation of β-amyloid plaques, and/or retarding the increase of amyloid load in the brain in a mammalian species in need thereof, comprising administering to the mammalian species a therapeutically effective amount of at least one compound according to claim 1 or a pharmaceutically acceptable salt thereof.

46. The method of claim 45, wherein the mammalian species is human.
